(19) United States
(12) Patent Application Publication
(10) Pub. No.: US 2021/0251572 A1
(43) Pub. Date: Aug. 19, 2021

(54) WEARABLE DATA COLLECTION DEVICE

(71) Applicant: SHIMMER RESEARCH LTD, Dublin (IE)

(72) Inventors: Geoffrey GILL, Cambridge, MA (US); Paddy WHITE, Dublin (IE); Mark NOLAN, Dublin (IE); Kathy HANLEY, Dublin (IE); Norman DAVIDSON, Dublin (IE)

(73) Assignee: SHIMMER RESEARCH LTD

(21) Appl. No.: 17/173,451

(22) Filed: Feb. 11, 2021

Related U.S. Application Data
(60) Provisional application No. 62/977,389, filed on Feb. 16, 2020.

(51) Int. Cl. A61B 5/00; H05K 5/02; G06F 1/16; G16H 40/67

(52) U.S. Cl. CPC A61B 5/6831; H05K 5/0213; G06F 1/163; G16H 40/67; A61B 5/0002; A61B 2560/0214; A61B 2560/0475; A61B 2562/227; A61B 2562/0219; A61B 2560/0443

(57) ABSTRACT

The wearable data collection device includes a processor, a sensor, a digital memory storage, and a battery. There can also be a data transmitter, a data transceiver, and a plurality of switches. The processor, sensor, digital memory storage, and any other electronics can be housed within a sensor module, while the battery is housed within a battery module. The modules are joined to one another to define a sealed unit to be fit in a housing. The housing can be attached by a strap to the user. The switches isolate the battery from the sensor, digital memory storage, the data transmitter, the data transceiver, or other electronic component.

### CROSS-REFERENCE TO RELATED APPLICATIONS

The present application claims priority under 35 U.S.C. Section 119(e) from U.S. Provisional Patent Application Ser. No. 62/977,329, filed on 16 Feb. 2020, entitled “DATA COLLECTION DEVICE”. See also Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present disclosure relates to a data collection device. In particular to such a device that may be worn by a user for collecting physiological data about the user.

### 2. Description of Related Art Including Information Disclosed Under 37 CFR 1.97 and 37 CFR 1.98

Real-time, non-invasive health monitors, which include one or more sensors integrated within small wearable devices are now widely known. Physiological data is collected by the devices for subsequent use. Data may, for example, be wirelessly transmitted or transmitted via a wired connection into a network, where the data is stored and/or processed. The information can be used to support a variety of useful methods, such as clinical trials, marketing studies, research studies, biofeedback, entertainment, and others.

Prior art devices that measure and/or collect physiological data typically suffer from one or more of a number of drawbacks, including but not limited to:
short battery life/the need to recharge the device after a short period of use;
the loss of data due to battery power loss; and
data being processed locally on the device and then discarded with only the processed results reported.

In prior art devices that require frequent recharging, the user has to remove the device for the period of recharging. Physiological data cannot be collected during the re-charging period, leading to a loss of valuable data. Moreover, there is a strong risk that the user may neglect to keep the device suitably charged leading to significant down time.

In prior art devices in which data is processed locally on the device and only the processed results are transmitted, the data cannot be re-processed or processed in a different manner. There is significantly less flexibility with the data collected.

The present invention arose in a bid to provide a more efficient device for collecting physiological data.

These and other objectives and advantages of the present invention will become apparent from a reading of the attached specification.

### BRIEF SUMMARY OF THE INVENTION

In one aspect, there is provided a wearable data collection device configured to be attached to a subject, comprising: a processor; at least one sensor; a digital memory storage; and at least one battery. The processor, sensor and digital memory storage are housed within a first module, and the battery is housed within a second module, the first and second modules are configured to be releasably joined to one another, wherein when they are joined to one another they define a sealed unit.

There is preferably further provided a housing that is configured to receive the sealed unit.

A strap is preferably attached to the wearable data collection device for suitable attachment to the subject. The strap is preferably attached to the housing. Other means of attaching the wearable data collection device to the subject may be employed.

The battery may be non-rechargeable. The battery may be irremovable from the second module. The second module may be disposable/replaceable.

In a further aspect, there is provided a wearable data collection device configured to be attached to a subject, comprising a housing for a battery, which comprises an air vent, the air vent being blocked by an air permeable and water impermeable material configured to allow airflow through the vent but prevent the ingress of water or other substances into the housing through the vent.

The battery is preferably a zinc air battery.

The air permeable and water impermeable material preferably comprises a porous material, which may comprise a porous membrane.

The air vent may be provided in a recess. The recess may comprise a channel. The recess acts to ensure an air path to the air vent is maintained. The recess is preferably formed in an outer a surface of the housing.

In a yet further aspect, there is provided a wearable data collection device configured to be attached to a subject, comprising: a processor; at least one battery; at least one sensor; a digital memory storage; at least one data transmitter or transceiver; and a plurality of switches. The switches are configured to be normally open for isolating the at least one sensor, the digital memory storage and the data transmitter or transceiver from the at least one battery.

The switches are under control of the processor, wherein they may be independently opened and closed to control the supply of battery power to any of the at least one sensor, the digital memory storage and the data transmitter or transceiver.

The switches preferably comprise physical switches. The switches may each comprise a transistor and a resistor. The switches may be closed by application of a current. The switches draw no current when open.

### BRIEF DESCRIPTION OF THE SEVERAL VIEWS OF THE DRAWINGS

FIG. 1 shows a side elevation view of the disassembled component parts of a data collection device according to one embodiment.

FIG. 2 shows a top plan view of the disassembled component parts of the embodiment of the data collection device in FIG. 1.

FIG. 3 shows a bottom plan view of the embodiment of the data collection device of FIG. 1 in an assembled form.

FIG. 4 shows a schematic view of the circuitry of a sensor module of an embodiment of the data collection device.

FIG. 5 shows a perspective view of embodiments of the sensor module and battery module.

FIG. 6 shows a perspective view of the embodiments of the sensor module and the battery module as a sealed unit.

FIG. 7 shows another perspective view of the embodiments of the sensor module and the battery module as a sealed unit with the vent recess at the peripheral edge.

FIG. 8 shows a bottom plan view of an embodiment of the housing.

FIGS. 9a and 9b show alternative side elevation views of the assembled data collection device.

FIGS. 10a and 10b show side and perspective views of another embodiment of the assembled data collection device with a wrist strap attached.

### DETAILED DESCRIPTION OF THE INVENTION

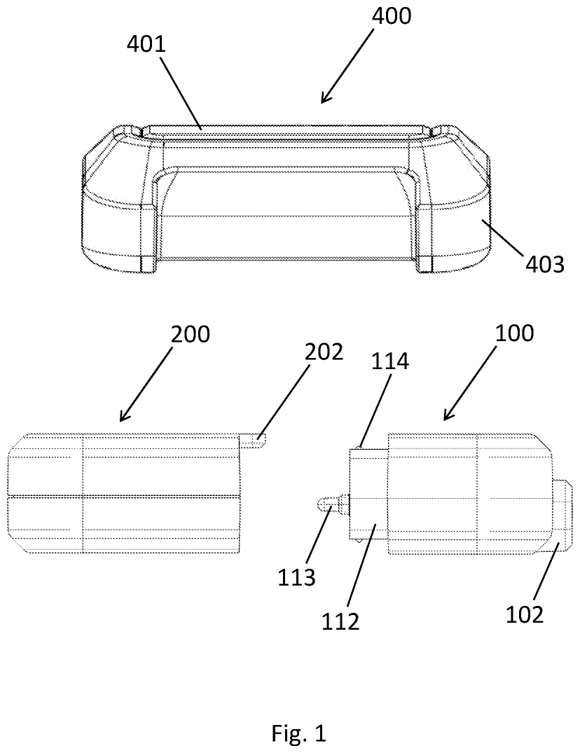

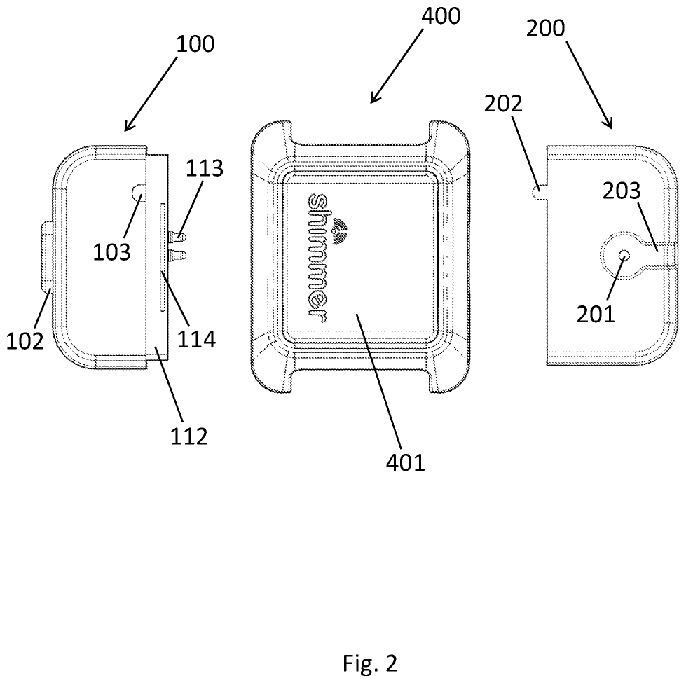

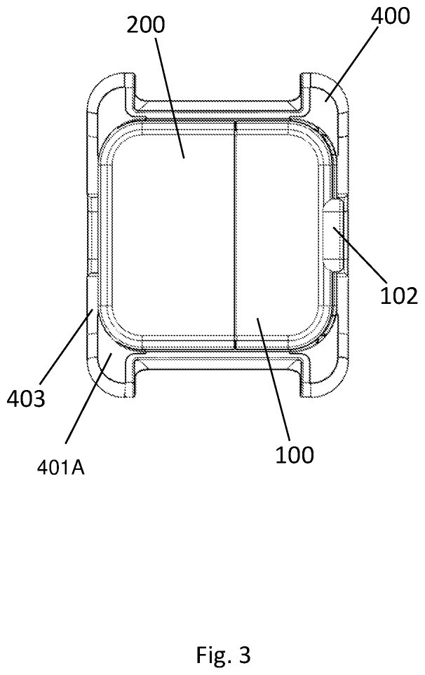

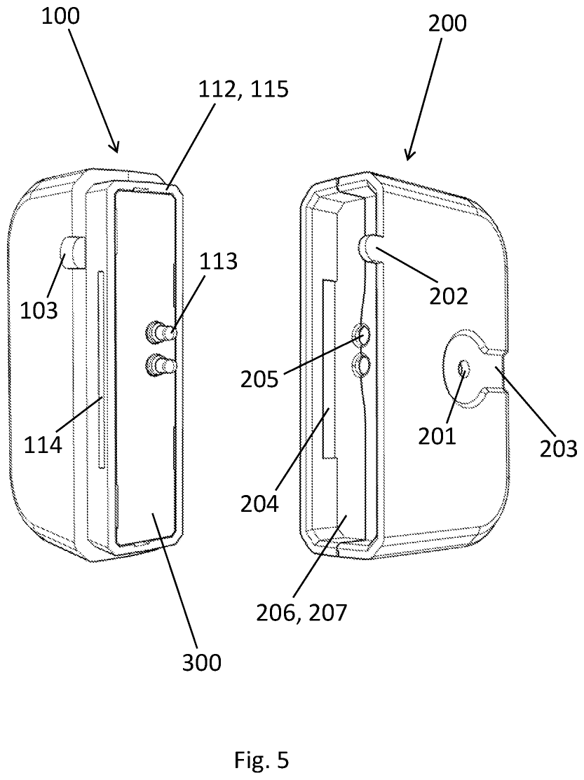

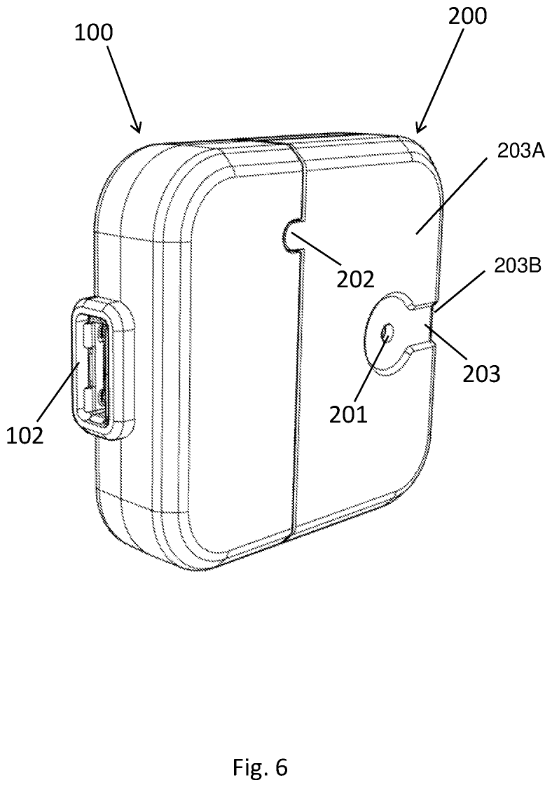

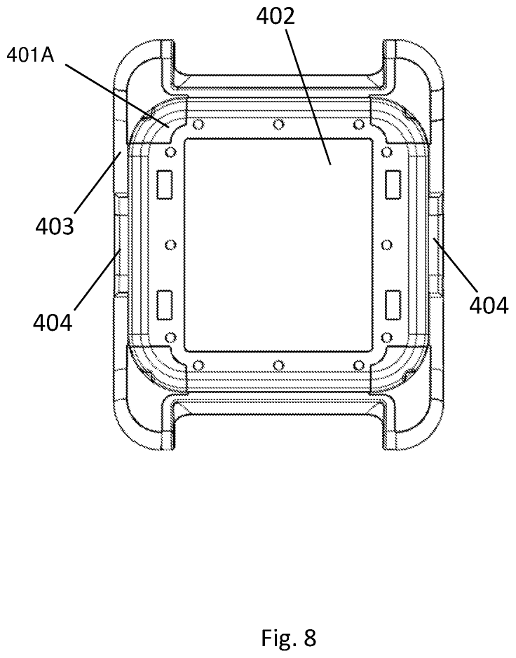

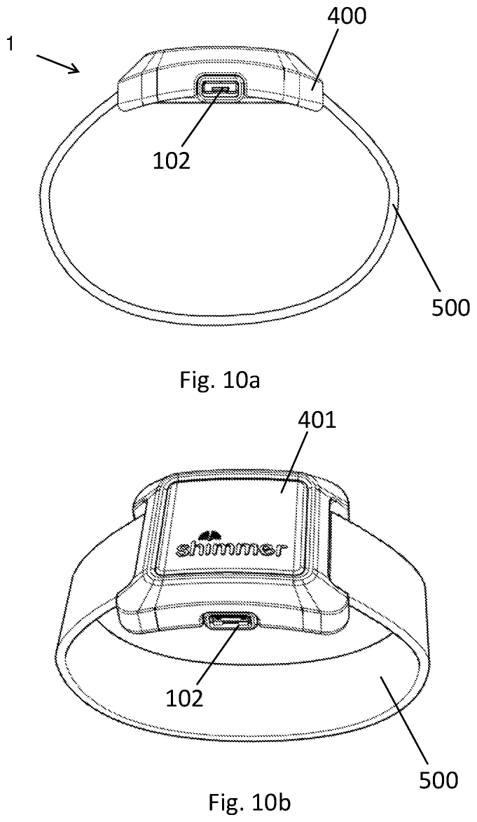

With reference to the FIGS. 1 to 3 and 5 to 10b, there is shown a data collection device. The data collection device 1 comprises an electronics module or sensor module 100, a battery module 200, and a housing 400. The sensor module 100 and the battery module 200 are configured to be combined with/connected to one another. The combined sensor module and battery module are received by the housing 400 (as best seen in FIGS. 3, 9a, 9b, 10a and 10c), which is provided with an attachment means for attaching the data collection device to a subject. The attachment means most preferably comprises a strap 500, as seen in FIGS. 10a and 10b. The strap may take the form of a wrist strap, an arm band, a chest band, a head band, a leg band, a foot band or any other band to allow of mounting of the device onto the subject. The attachment means could, however, alternatively comprise an adhesive pad, clip, or otherwise, as will be readily appreciated by those skilled in the art.

In alternative arrangements, the electronics module or sensor module 100 and the housing 400 could be integrated with one another with the battery module 200 attachable and detachable therefrom.

In the present arrangement, the housing 400 has an outer face 401, an inner face 401A opposite the outer face 401 so as to form an open back. There is a recess 402 defined that is suitably sized and shaped for receiving the combined sensor module 100 and battery module 200. The housing 400 preferably substantially encloses the combined sensor module 100 and battery module 200. The housing 400 preferably has a closed upper (or outer) face 401, such that when the data collection device is worn by a subject with the enclosed combined sensor module 100 and battery module 200 facing the subject's body it is substantially concealed and inaccessible. The sensor module preferably comprises the closed upper (or outer) face 401 and a peripheral wall 403 that depends therefrom and defines the recess 402. The upper face 401 may be substantially planar or may be curved or otherwise formed. It should be noted, however, that the recess may be formed otherwise, i.e., in alternative arrangements it need not be formed by a peripheral wall. The combined sensor module 100 and battery module 200 preferably snap fits into the recess to be suitably held in place within the housing. It may be held in place by an interference fit. The peripheral wall 403 preferably has suitable cut outs or openings 404 for providing access to data ports 102 and/or to provide an air channel for the battery 120, in particular via vent recess 203, as discussed below.

The combined sensor module 100 and battery module 200 preferably defines a sealed unit, which is sealed against the ingress of moisture to render it waterproof. There is a suitable mechanical/electrical interconnection between the sensor module 100 and the battery module 200. In the present arrangement, the sensor module 100 comprises a sensor module electrical connector 113 as a male connector for receipt by a batter module electrical connector 205 as a female connector of the battery module 200. In alternative arrangements the male and female connectors could be swapped with one another. There is a resilient sealing element 300 or gasket between the sensor module 100 and the battery module 200 to allow for sealing.

Considering the mechanical/electrical interconnections and the sealing in the present exemplary arrangement in more detail:

The electronics module or sensor module 100 comprises a sensor module face 112 provided with one or more sensor module electrical connectors 113 and the battery module 200 comprises a battery module face 206 provided with one or more battery module electrical connectors 205, configured to connect with the sensor module electrical connectors 113. The sensor module electrical connectors 113 comprise pins, which may be spring loaded. The battery module electrical connectors 205 comprise recessed contacts for receiving the pins. As will be readily appreciated various other forms of electrical connector could be implemented instead. The sensor module face 112 is provided with the gasket 300. However, the resilient sealing member need not be limited to a gasket. It may take various alternative forms. It is most preferably resilient.

The sensor module face 112 or first face is the face of a plug portion 115 and the battery module face 206 or second face is the face of a socket portion 207, which is configured to receive the plug portion 115. The plug portion is provided with at least one protrusion 114 (protruding substantially perpendicular to an insertion direction of the plug) and the socket is provided with at least one corresponding socket recess 204, which is configured to receive the protrusion when the plug portion 115 is received by the socket 207 portion. The protrusion 114 and socket recess 204 provide an interlock. In alternative arrangements, the protrusion 114 and socket recess 204 could be omitted.

The configuration is such that when the faces 112 and 206 are brought together to form the electrical connection (between the electrical connectors 113 and 205), and the sealed unit is then inserted into the recess of the housing 400, the resilience of the gasket 300 causes an interference fit between the sealed unit and the recess 402 of the housing 400. The interference fit is provided as follows: pressing the sealed unit into the recess 402 causes a compression of the gasket 300, wherein the gasket urges outer faces of the sensor module 100 and the battery module 200, respectively, against opposed faces of recess 402 defined by the peripheral wall 403. The width of the socket recess 204 may be arranged to be sufficient to accommodate limited expansion of the gasket 300 prior to insertion of the sealed unit into the socket recess 402.

It should be appreciated that any of the paired mechanical and/or electrical connectors, as detailed above could be reversed between the two modules, as desired.

To ensure the sensor module 100 cannot be connected to the battery incorrectly and/or to provide a clear indication to the subject of the correct orientation, a locating tab 202 is preferably provided that fits in a locating recess 103. The locating tab 202 and the locating recess 103 may be swapped over in alternative arrangements. In further alternative arrangements they may be omitted.

The combined sensor module 100 and battery module 200 is further waterproof by the use of a waterproof wired connector 102.

The data collection device is capable of collecting physiological raw data, storing the raw data locally on the device and transferring the raw data for subsequent analysis. The transfer may be affected by means of a wireless or wired connection. The data may be stored locally on the device for any suitable period. It may be stored locally for several months.

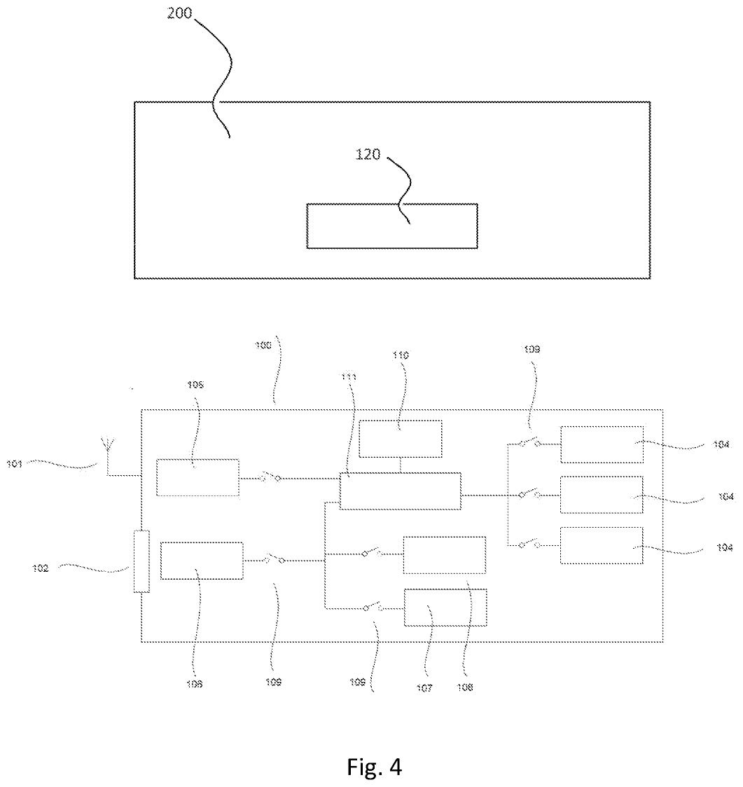

Raw data in this context is data that has been sensed by one or more sensors (104, FIG. 4). The sensed data most preferably comprises physiological data. The data is raw in the sense that there is no local processing of the data on the data collection device in any manner whatsoever. The sensed data is stored on the device in raw, unprocessed, form. The device is configured to transfer the raw data for processing off the device.

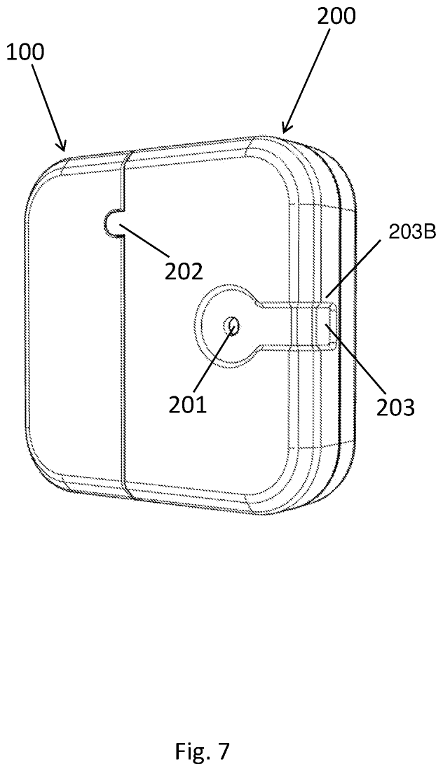

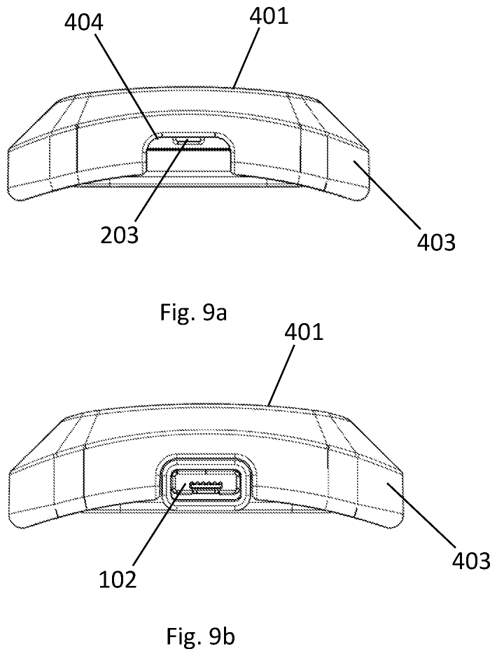

The battery module 200 houses one or more batteries (120, FIG. 4) for powering the sensor module 100. The battery 120 is most preferably non-rechargeable and capable of continuously powering the sensor module 100 for a number of months, most preferably up to at least 6 months. The one or more batteries 120 may comprise Zinc Air batteries. Zinc Air batteries require air to operate. For such purpose, an air vent 201 is included in the battery module. The air vent is preferably waterproof, configured to allow the passage of air through the vent 201 but to prevent the ingress of moisture into the battery module 200. For this purpose, the air vent 201 comprises an air permeable and water impermeable material. The air vent may, for example, comprise a GAW337 or GAW338 acoustic vent from W. L Gore. The air vent 201 may be provided in a vent recess 203 that acts to define an air passage. In the present arrangement, the vent recess 203 comprises an elongate channel. The air vent 201 is preferably provided in an upper surface or outer battery surface 203A of the housing of the battery module 200. The outer battery surface 203A faces the inner face 401A of the housing 400 when the data collection device is worn by a subject. The vent recess 203 extends at least to a peripheral edge 203B of the upper surface. Such an arrangement ensures that the air vent cannot be blocked by the subject. In the present arrangement, as best seen in FIG. 7, the vent recess 203 extends to the peripheral edge 203B of the upper surface or outer battery surface 203A of the housing of the battery module and across a peripheral face thereof, which is substantially perpendicular to the upper surface or outer battery surface 203A. The vent recess 203 is in alignment with a suitable cut out or opening 404 in the peripheral wall 403 of the housing 400, as mentioned above, and as best seen in FIG. 9a.

It should be noted that whilst it is preferable that the battery 120 comprises a non-rechargeable/single use/disposable battery, it may alternatively comprise a suitable rechargeable battery.

The circuitry and operation of the data collection device will now be considered with particular reference to FIG. 4.

FIG. 4 shows, schematically, the sensor module 100 and the battery module 200. It is to be noted that the electrical interconnection between the modules is omitted from FIG. 4.

The sensor module 100 comprises a power supply module 110, a processor 111, a plurality of sensors 104, a digital memory, which comprises a raw data storage device 107 and a configurable setting storage device 108, a wireless data module 105, a wired data module 106, a wired connector 102 and a wireless antenna 101. A plurality of switches 109 are provided for isolating different elements from the power supply module 110 for limiting power draw. The power supply module 110 is in removable connection with the battery 120 in the battery module 200.

It is to be noted that any suitable physical implementation may be put into effect. The processor, for example, may comprise a suitable system-on-chip.

The device may contain one or more sensors 104, including any combination of the following sensors: Accelerometer, Gyroscope, Magnetometer, Inclinometer, Temperature, Galvanic Skin Response (GSR), Electroencephalogram (ECG), Electromyography (EMG), Photoplethysmograph (PPG), Electroencephalogram (EEG), Electrooculography (EOG), Bio-Impedance, Pressure Sensor, Light Sensor, Humidity, Respiration, and Gas/Air.

In one exemplary arrangement, there is only an accelerometer and a gyroscope provided.

As mentioned, in the present arrangement, the digital memory comprises a raw data storage device 107 and a configurable setting storage device 108. The raw data storage device may comprise a flash memory, allowing for blank erase. The configurable setting storage device 108 may comprise an electrically erasable programmable read-only memory (EEPROM).

The wireless connection module 105 may comprise a Wi-Fi module, a Bluetooth module, or otherwise.

The switches 109 comprise physical switches. They may each comprise a transistor and a resistor and be configured such that they are normally open. With the switches normally open portions of the circuit are isolated from the power supply module 110 by default. The switches will close with an applied current. They draw no current in their open state. Various suitable switching solutions will be readily conceived by those skilled in the art.

The arrangement is such that if any portion of the device is not in use at a given time, rather than placing the section in standby or low power mode, the device removes power to the relevant component/module/section of the circuit. This offers a significant reduction in power consumption, as compared to a system in which a standby or low power mode is implemented.

The power to the wireless data module 105 can be turned off when the wireless data module 105 is not in use; the power to the wired data module 106 can be turned off when the wired connection 102 is not in use; the power to the raw data storage device 106 can be turned off when raw data is not being written, read or erased; the power to the configurable setting storage device 107 can be turned off when the configuration is not being read, written, or updated; and the power to one or more of the sensors 104 can be turned off when not in use. In some embodiments collecting data with the least power, at least one switch 109 is closed between the sensor 104 and the processor 111, and at least another switch 109 is closed between the processor 111 and the raw data storage 107.

As a further power saving feature, at any time when the device is connected via a wired link, such as USB, to the wired connector 102, the device is configured to automatically switch from battery power to the wired power source. The wired connector 102 is waterproof to ensure sealing of the sensor module 100.

Power usage (and thereby battery life) is optimized by removing power to the different sections of the electronics module 100 when they are not being used, rather than placing the sections in standby or low power mode, as is common in prior art arrangements.

A significant advantage of the present data collection device is its low maintenance and long battery life. As discussed, it may be powered continuously for a number of months with no action required by the subject.

A preferred data transfer arrangement is now considered.

When transferring the raw data wirelessly, the power usage is minimized by transferring the stored raw data at a pre-configured interval. The interval may be set as appropriate and is not particularly limited. It may be daily, every other day, weekly, monthly, or otherwise. Outside the pre-configured interval, the device powers down the wireless section of the device by the opening of the appropriate switch 109.

The time of day for the transferring of raw data can also be configured. Non-limiting examples of configurable times are daily at 1:00 am local time or every two days at 9:00 pm. Clearly numerous alternative times/intervals may be selected.

The device is capable of adapting its transfer interval and time such that data is wirelessly transmitted when in range of an appropriate transceiver, which may comprise a smart phone of the subject. If the device cannot establish a connection to wirelessly transfer the raw data after a configurable number of attempts, the device is configured to vary its connection time. The device starts from the configured time and consecutively increments its connection time by a configurable number of minutes until a connection is established. The time at which the wireless connection is established is then used as the new wireless connection time. For example, if the device is configured to transfer the raw data daily at 1:00 am and cannot establish a wireless connection to transfer the raw data after 3 days, the connection time is incremented by 7.1 hours to 8:06 am. The device attempts to establish a wireless connection at the new time. If the device successfully establishes a wireless connection then the new time is used, if a wireless connection cannot be established the connection time is further incremented by 7.1 hours to 3:12 pm. The time increment is configurable.

The raw data is continuously collected by the device and stored. That is, at least one switch 109 is closed between the sensor 104 and the processor 111, and at least another switch 109 is closed between the processor 111 and the raw data storage 107. At the configurable time and date, the wireless connectivity portion of the section is powered and activated. The device then retrieves the raw data from storage and transmits it. That is, at least one switch is closed between the wired data module and the processor, and at least another switch is closed between the processor and the raw data storage. After confirmation that the data has been successfully transferred the wireless connectivity portion of the device is powered down. Also, after confirmation that the data has been successfully transferred the data is erased from the local storage, freeing the storage to be re-used.

The foregoing disclosure and description of the invention is illustrative and explanatory thereof. Various changes in the details of the illustrated structures, construction and method can be made without departing from the true spirit of the invention.

We claim:

1. A wearable data collection device, comprising:
a sensor module;
a processor housed within said sensor module;
a sensor housed within said sensor module;
a digital memory storage housed within said sensor module;
a battery module removably attached to said sensor module so as to form a sealed unit; and
a battery housed within said battery module.

2. The wearable data collection device, according to claim 1, further comprising:
a housing having an outer face, an inner face opposite said outer face, a peripheral wall around said inner face so as to form a recess, said sealed unit being removably attached within said recess.

3. The wearable data collection device, according to claim 2, further comprising:
a strap removably attached to said housing.

4. The wearable data collection device, according to claim 2,
wherein said sensor module is comprised of: a sensor module face with a sensor module electrical connector,
wherein said battery module is comprised of: a battery module face with a battery module electrical connector, and
wherein said sensor module face faces toward said battery module face, said sensor module electrical connector being removable connected to said battery module electrical connector, the device further comprising:
a resilient sealing element between said sensor module face and said battery module face.

5. The wearable data collection device, according to claim 4, wherein said resilient sealing element is comprised of a gasket.

6. The wearable data collection device, according to claim 4, wherein said sensor module electrical connector is a male connector, and wherein said battery module electrical connector is a female connector cooperative with said male connector.

7. The wearable data collection device, according to claim 6, wherein said sensor module face is comprised of a plug portion, and wherein said battery module face is comprised of a socket portion, said plug portion and said socket portion being in removable male-female engagement.

8. The wearable data collection device, according to claim 4, wherein said resilient sealing member is in removable interference fit engagement with said recess.

9. The wearable data collection device, according to claim 4, wherein said sensor module face is further comprised of a locating recess, and wherein said battery module face is further comprised of a locating tab, said locating tab and said locating recess being in removable male-female engagement so as to align said sensor module and said battery module in said sealing unit.

10. The wearable data collection device, according to claim 1, wherein said battery module is comprised of an air vent being comprised of an air permeable and water impermeable material.

11. The wearable data collection device, according to claim 10, wherein said air permeable and water impermeable material is comprised of a porous material.

12. The wearable data collection device, according to claim 10, further comprising:
a housing having an outer face, an inner face opposite said outer face, a peripheral wall around said inner face so as to form a recess, said sealed unit being removably attached within said recess,
wherein said battery module is comprised of:
an outer battery surface facing said inner face of said housing; and
a vent recess on said outer battery surface, said air vent being in fluid connection with said vent recess.

13. The wearable data collection device, according to claim 12, wherein said vent recess extends to a peripheral edge of said battery module, said air vent being in fluid connection with said peripheral edge.

14. The wearable data collection device, according to claim 12, wherein said vent recess is comprised of an elongate channel.

15. The wearable data collection device, according to claim 12, wherein said peripheral wall is comprised of openings, and wherein said vent recess aligns with at least one opening, said air vent being in fluid connection through said vent recess and said at least one opening.

16. A wearable data collection device 1, comprising:
a sensor module;
a power supply module housed within said sensor module;
a processor housed within said sensor module;
a sensor housed within said sensor module;
a digital memory storage housed within said sensor module;
a data transmitting and receiving device housed within said sensor module;
a plurality of switches housed within said sensor module;
a battery module removably attached to said sensor module so as to form a sealed unit; and
a battery housed within said battery module and being in removable connection with said power supply module.

17. The wearable data collection device, according to claim 16, wherein said digital memory storage is comprised of a raw data storage device and a configurable setting storage device.

18. The wearable data collection device, according to claim 17, wherein at least one switch is closed between said sensor and said processor, and wherein at least another switch is closed between said processor and said raw data storage.

19. The wearable data collection device, according to claim 16, further comprising:
a wireless data module housed within said sensor module; and
a wired data module housed within said sensor module.

20. The wearable data collection device, according to claim 19, wherein at least one switch is closed between said wired data module and said processor, and wherein at least another switch is closed between said processor and said raw data storage.
